(19) United States
(12) Patent Application Publication
(10) Pub. No.: US 2026/0354916 A1
(43) Pub. Date: Nov. 12, 2026

(54) NAVIGATION SYSTEM FOR PREOPERATIVE PLANNING AND INTRAOPERATIVE MONITORING IN THE TREATMENT OF CHRONIC OSTEOMYELITIS

(71) Applicant: The Fourth Medical Center of the Chinese People's Liberation Army General Hospital, Beijing (CN)

(72) Inventors: Jianzheng ZHANG, Beijing (CN); Jianwen ZHAO, Beijing (CN); Hongying HE, Beijing (CN); Huhu WANG, Beijing (CN); Juqiang HAN, Beijing (CN); Qing LI, Beijing (CN); Ming HAO, Beijing (CN); Bo WANG, Beijing (CN); Weidong SHI, Beijing (CN); Tiansheng SUN, Beijing (CN); Peifu TANG, Beijing (CN)

(73) Assignee: The Fourth Medical Center of the Chinese People's Liberation Army General Hospital, Beijing (CN)

(21) Appl. No.: 19/670,241

(22) Filed: May 7, 2026

(30) Foreign Application Priority Data
May 8, 2025 (CN) 202510585323.X

(51) Int. Cl. A61B 34/32; A61B 90/35; G16H 20/40; A61B 34/10; A61B 34/20; A61B 90/50

(52) U.S. Cl. CPC A61B 34/32; A61B 90/35; G16H 20/40; A61B 2034/105; A61B 2034/2057; A61B 2034/2065; A61B 2090/504

(57) ABSTRACT

A navigation system for preoperative planning and intraoperative monitoring in the treatment of chronic osteomyelitis includes a vertical arm. A wound auxiliary positioning disc is disposed on one side of the top end of the vertical arm, a camera is disposed at a central position of the wound auxiliary positioning disc, wound auxiliary positioning lamps are disposed on a lower surface of the wound auxiliary positioning disc, a plurality of the wound auxiliary positioning lamps are evenly arranged around the camera, and each of the wound auxiliary positioning lamps emits a monochromatic light beam. A support rail is disposed on the vertical arm, a robotic manipulator is mounted on the support rail, and a clamping assembly capable of performing a clamping action is disposed at an end of the robotic manipulator.

### CROSS-REFERENCE TO RELATED APPLICATIONS

This application claims priority to Chinese Patent Application No. 202510585323.X, filed on May 8, 2025, the content of which is incorporated herein by reference in its entirety.

### TECHNICAL FIELD

The present application belongs to the field of medical devices, and more particularly to a navigation system for preoperative planning and intraoperative monitoring in the treatment of chronic osteomyelitis.

### BACKGROUND

At present, conventional debridement procedures are generally plagued by insufficient accuracy in lesion localization, limited intraoperative visualization, and low operational efficiency. Preoperative planning based on two-dimensional imaging such as CT and MRI fails to intuitively display the three-dimensional spatial relationships of lesions. Repeated confirmation by palpation or fluoroscopy is required during surgery, resulting in an average localization error at the centimeter level. With the continuous reduction of incisions in minimally invasive surgery, deep lesions are difficult to expose. Surgeons have to judge tissue layers based on experience, which increases the risk of damage to vital anatomical structures. Debridement in complex anatomical areas including the skull base and spine requires frequent adjustment of surgical instrument angles, leading to poor surgical efficiency.

As a cutting-edge achievement of the interdisciplinary integration of modern medicine, surgical navigation technology has been widely applied in surgical fields including neurosurgery, orthopedics and otorhinolaryngology. Nevertheless, although existing surgical navigation systems can assist surgeons in operations, they have deficiencies in preoperative planning and intraoperative monitoring, and carry risks of surgical deviation and operational errors. To solve the above defects, the present application proposes a navigation system for preoperative design and intraoperative monitoring in the treatment of chronic osteomyelitis.

On the basis of this, it is the object of the present application to provide a navigation system for preoperative planning and intraoperative monitoring in the treatment of chronic osteomyelitis, so as to solve the technical problem of risks of surgical deviation and operational errors existing in current debridement procedures.

To achieve the above objectives, the present application provides the following technical solutions: A navigation system for preoperative planning and intraoperative monitoring in the treatment of chronic osteomyelitis, comprising a vertical arm, wherein: a wound auxiliary positioning disc is disposed on one side of the top end of the vertical arm, a camera is disposed at a central position of the wound auxiliary positioning disc, a plurality of wound auxiliary positioning lamps are disposed on a lower surface of the wound auxiliary positioning disc, each of the wound auxiliary positioning lamps emits a monochromatic light beam, and the wound auxiliary positioning lamps are evenly arranged around the camera, a support rail is disposed on the vertical arm, a robotic manipulator is mounted on the support rail, and a clamping assembly capable of performing a clamping action is disposed at an end of the robotic manipulator.

The present application is further arranged such that the plurality of the wound auxiliary positioning lamps are divided into at least two groups, the wound auxiliary positioning lamps of different groups emit light of different colors, and the groups of wound auxiliary positioning lamps are distributed at intervals.

The present application is further configured such that the vertical arm comprises a vertically disposed outer arm and an inner arm movably extending through the top end of the outer arm, a second pen-type electric push rod is vertically fixed inside the outer arm, a receiving cavity for receiving the second pen-type electric push rod is formed at the bottom end inside the inner arm, and the telescopic end of the second pen-type electric push rod is connected to the inner top wall of the receiving cavity.

The present application is further configured such that a counterweight base is disposed at a bottom end of the outer arm, universal wheels are disposed at four corners of the bottom of the counterweight base, and the counterweight base is formed as a frustoconical structure.

The present application is further configured such that a connecting plate is horizontally fixed to one side of the wound auxiliary positioning disc, a fixing sleeve is fixed to an end of the connecting plate, the fixing sleeve is fitted over the top end of the inner arm and fixed to the inner arm by bolts, a reinforcing plate is fixed between an upper surface of the connecting plate, an upper surface of the wound auxiliary positioning disc, and a side wall of the fixing sleeve and the reinforcing plate is formed as a triangular structure.

The present application is further configured such that the support rail is horizontally fixed to one side of the outer arm, an adjustment arm is movably embedded on the support rail in a length direction thereof, the adjustment arm extends through an end of the support rail toward the wound auxiliary positioning disc, the robotic manipulator is mounted on the adjustment arm, a lead screw is rotatably mounted within the support rail, the adjustment arm is threadedly engaged with the lead screw, a second manipulation motor for driving the lead screw to rotate is mounted at an end of the support rail, a guide groove is formed in each of two side walls of the adjustment arm in the length direction thereof, and a protrusion is disposed on each of two inner side walls of the support rail, each protrusion is engaged with a corresponding guide groove.

The present application is further configured such that the robotic manipulator comprisesa fixing base fixed on the adjustment arm, a first manipulation arm horizontally and rotatably mounted on the fixing base, a second manipulation arm longitudinally and rotatably mounted on the first manipulation arm, a third manipulation arm longitudinally and rotatably mounted on the second manipulation arm, and a fourth manipulation arm longitudinally and rotatably mounted on the third manipulation arm, a first manipulation motor is disposed at a rotating shaft of each of the first manipulation arm, the second manipulation arm, the third manipulation arm and the fourth manipulation arm, each first manipulation motor is capable of driving its corresponding manipulation arm to perform horizontal rotation and longitudinal rotation., and the clamping assembly is disposed at the bottom end of the fourth manipulation arm.

The present application is further configured such that the clamping assembly comprises a connection head rotatably mounted at the bottom end of the fourth manipulation arm, and a pair of slide rails horizontally and slidably embedded at the bottom of the connection head, the pair of slide rails are arranged side by side, a clamping head is disposed at the bottom of each slide rail, the pair of clamping heads are symmetrically disposed, a mounting hole is formed in each clamping heads, and a first pen-type electric push rod for driving the slide rails to move horizontally is mounted on the connection head.

The present application is further configured such that a control box is disposed on a side wall of the outer arm, a circuit board and a chip are arranged inside the control box, an intelligent control system is arranged based on the circuit board and the chip, the intelligent control system comprises: an image acquisition module is configured to capture, via the camera, image information of a patient's wound, comprising details such as shape, size, and depth of the wound, thereby providing an accurate data foundation for preoperative debridement assessment and localization of the patient, the image acquisition module supporting a multi-angle shooting function to obtain complete wound information from different perspectives, allowing comprehensive understanding of wound conditions and providing support for formulating a more reasonable debridement plan; a wireless data transmission control module is configured to employ wireless transmission technology, such as GPRS, to transmit image data captured by the camera in real time to an information management center server, the information management center server has at least one IP address and stores an electronic human diagram, and upon receiving the image data, the server performs data processing and storage using TCP/IP protocol, thereby providing support for subsequent evaluation and analysis; an artificial intelligence assessment module is configured to, based on AI technology, analyze the captured wound images and automatically assess the extent of the wound, comprising a wound area assessment system and a wound color assessment system, through AI recognition, patient wound conditions are recorded and archived in a timely manner, based on the extent and characteristics of the wound, the system uses an artificial intelligence algorithm to determine key debridement areas, the system simulates the debridement process, reconstructs the wound and surrounding tissues using three-dimensional (3D) modeling technology, and performs marked display based on the electronic human diagram, allowing a surgeon to perform debridement operations in a virtual environment, evaluate debridement effects in advance, and optimize a surgical plan; and a robotic arm assistance module is configured to employ a robotic manipulator as an auxiliary device, in orthopedic surgery, a surgeon uses fluoroscopy or scanning to input a patient's 3D body information into a computer, after the system automatically completes matching, the surgeon can select an appropriate surgical path, and once the surgical path is confirmed, the robotic manipulator accurately moves to a specified position with sub-millimeter accuracy through camera navigation and multi-degree-of-freedom motion control technology, synchronizes with a surgical tool to be introduced into the patient's body, and achieves plate fixation of bone.

In summary, the present application has the following beneficial effects:

Through the provision of the camera, the present application can clearly capture image information of a patient's wound, including details such as the shape, size, and depth of the wound, thereby providing an accurate data foundation for subsequent evaluation and localization, the multi-angle shooting function of the camera can obtain complete wound information from different perspectives, allowing a more comprehensive understanding of the wound condition and providing support for formulating a more reasonable debridement plan, after acquiring the wound image information via the camera, a doctor can comprehensively examine the patient's wound before surgery, determine the specific location of the wound, assess the extent of the wound, and identify the key areas for debridement, and after determining the wound extent and identifying the key debridement areas, multiple groups of wound auxiliary positioning lamps can be controlled to emit light beams of different colors onto the patient's wound, thereby distinguishing and marking the wound extent and the key debridement areas, which allows the doctor to perform surgical operations more precisely, reducing surgical time and trauma.

Through the provision of the robotic manipulator and the clamping assembly, the present application controls the robotic manipulator to move the clamping assembly accurately to a designated position and synchronize it with the surgical tool to be introduced into the patient's body, thereby achieving a bone plate clamping and fixation action, which ensures that the plate fits closely to the bone and provides a stable fixation effect, such close fitting and stable fixation help promote fracture healing and reduce the occurrence of complications, and by controlling the first manipulation motors, the corresponding first, second, third, and fourth manipulation arms can be driven to perform horizontal rotation and longitudinal rotation movements respectively, coupled with the horizontal movement adjustment of the adjustment arm, the multi-degree-of-freedom adjustment control helps improve surgical efficiency, shorten surgical time, simplify the surgical operation process, and reduce the doctor's workload.

Through the provision of the control box, the present application utilizes artificial intelligence algorithms to simulate the debridement process and formulate surgical plans, allowing the doctor to evaluate debridement effects in advance, optimize the surgical plan, reduce surgical risks, and improve surgical precision and outcomes, furthermore, intelligent control enables telemedicine, and the wireless data transmission control function allows doctors to evaluate the patient's wound and guide surgery remotely, providing technical support for telemedicine.

### BRIEF DESCRIPTION OF THE DRAWINGS

FIG. 1 is a schematic structural view of the present application.

FIG. 2 is a schematic structural view of the wound auxiliary positioning disc of the present application.

FIG. 3 is a schematic bottom structural view of the wound auxiliary positioning disc of the present application.

FIG. 4 is a schematic structural view of the robotic manipulator of the present application.

FIG. 5 is a schematic structural view of the clamping assembly of the present application.

FIG. 6 is a schematic structural view of the support rail of the present application.

FIG. 7 is a schematic internal structural view of the outer arm of the present application.

### DETAILED DESCRIPTION OF THE EMBODIMENTS

The technical solutions of the embodiments of the present application will be described clearly and completely below with reference to the accompanying drawings. The embodiments described below with reference to the accompanying drawings are exemplary, merely intended to illustrate the present application, and shall not be construed as limiting the present application.

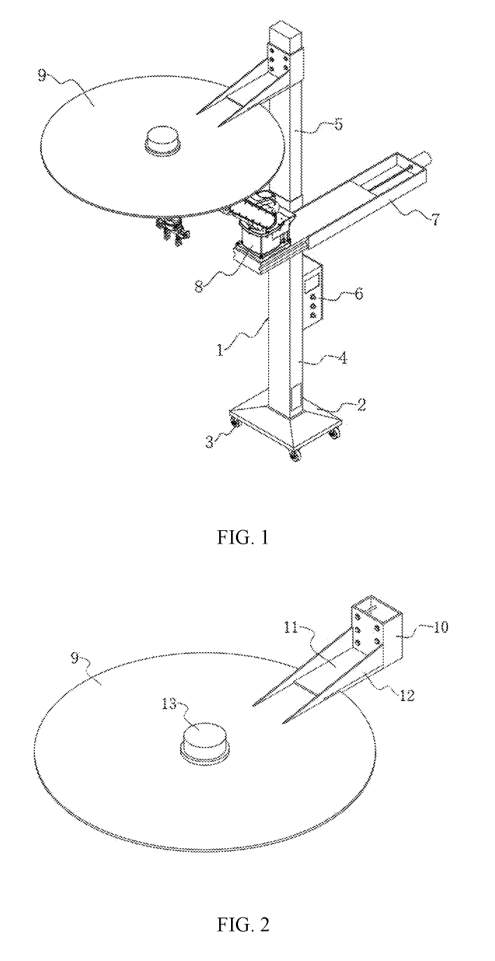

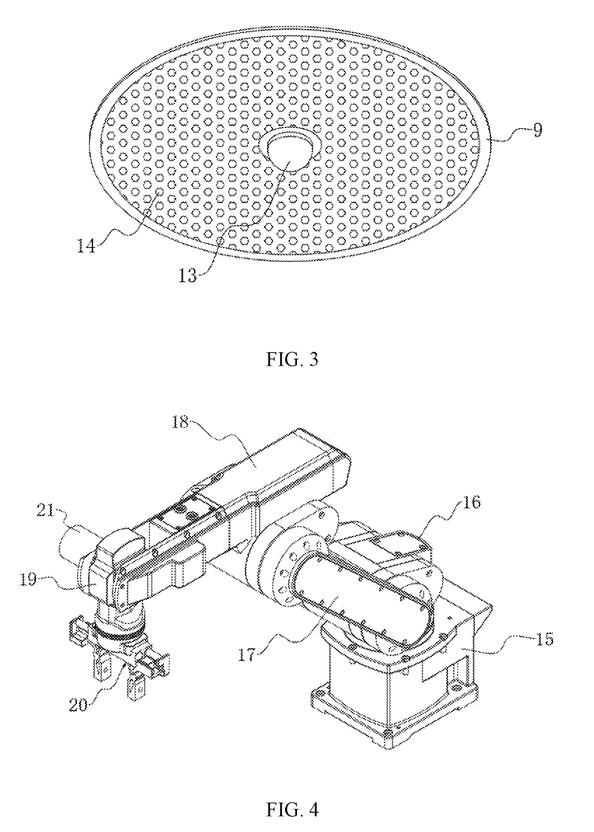

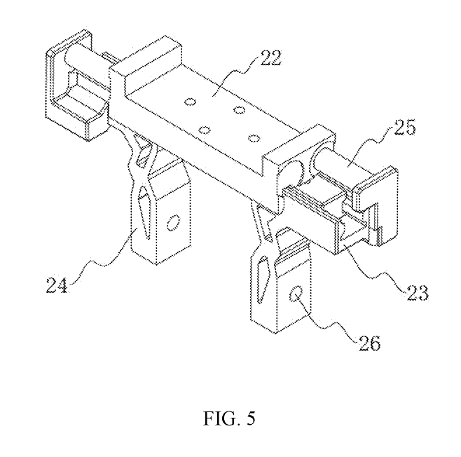

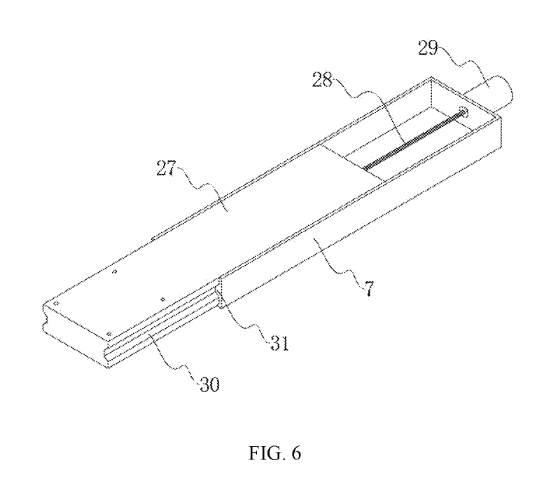

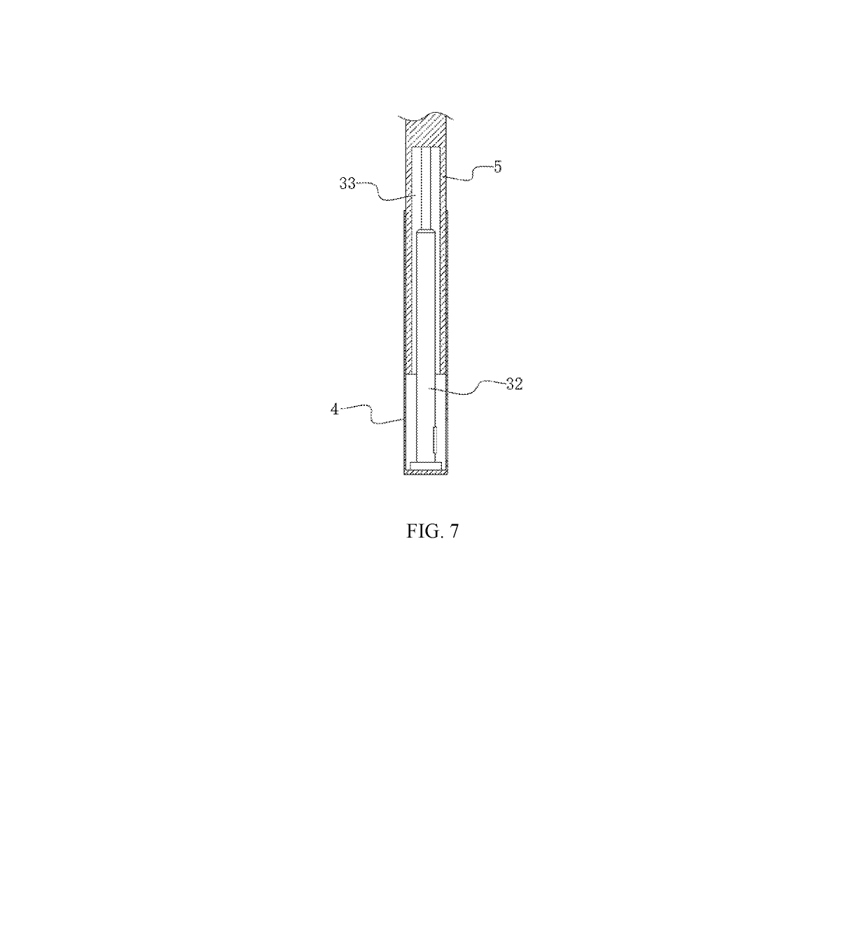

A navigation system for preoperative planning and intraoperative monitoring in the treatment of chronic osteomyelitis, as shown in FIGS. 1-7, comprises a vertical arm 1. A wound auxiliary positioning disc 9 is disposed on one side of the top end of the vertical arm 1. A camera 13 is disposed at a central position of the wound auxiliary positioning disc 9. Wound auxiliary positioning lamps 14 are disposed on a lower surface of the wound auxiliary positioning disc 9. A plurality of the wound auxiliary positioning lamps 14 are evenly arranged around the camera 13, and each of the wound auxiliary positioning lamps 14 emits a monochromatic light beam. The plurality of the wound auxiliary positioning lamps 14 are divided into at least two groups, the wound auxiliary positioning lamps 14 of different groups emit light of different colors, and the groups of wound auxiliary positioning lamps 14 are distributed at intervals. The camera 13 is a high-resolution camera capable of clearly capturing image information of a patient's wound, including details such as shape, size, and depth, thereby providing an accurate data foundation for subsequent evaluation and localization. Furthermore, the camera 13 supports a multi-angle shooting function to obtain complete wound information from different perspectives, avoiding evaluation deviation caused by a single perspective. Through multi-angle shooting, the wound condition can be understood more comprehensively, providing support for formulating a more reasonable debridement plan. After acquiring the wound image information via the camera 13, a doctor can comprehensively examine the patient's wound before surgery, determine the specific location of the wound, and assess the extent of the wound, including its length, width, and depth, which helps the doctor judge the difficulty of debridement and the amount of devitalized tissue and foreign matter to be removed. Based on the determined wound extent, the doctor can formulate a debridement plan and identify the key debridement areas, including determining which tissues need to be removed, which tissues need to be preserved, and how to handle contamination and foreign matter around the wound. After determining the wound extent and identifying the key debridement areas, the multiple groups of wound auxiliary positioning lamps 14 can be controlled to emit light beams of different colors onto the patient's wound, thereby distinguishing and marking the wound extent and the key debridement areas, which allows the doctor to perform surgical operations more precisely, reducing surgical time and trauma. In addition, during the surgical debridement process, the camera 13 can perform real-time intraoperative monitoring to ensure surgical accuracy.

In a further embodiment, the vertical arm 1 comprises a vertically disposed outer arm 4 and an inner arm 5 movably extending through the top end of the outer arm 4. A second pen-type electric push rod 32 is vertically fixed inside the outer arm 4. A receiving cavity 33 for receiving the second pen-type electric push rod 32 is formed at the bottom end inside the inner arm 5, and the telescopic end of the second pen-type electric push rod 32 is connected to the inner top wall of the receiving cavity 33. A connecting plate 11 is horizontally fixed to one side of the wound auxiliary positioning disc 9. A fixing sleeve 10 is fixed to an end of the connecting plate 11. The fixing sleeve 10 is fitted over the top end of the inner arm 5 and fixed to the inner arm 5 by bolts. A reinforcing plate 12 is fixed between an upper surface of the connecting plate 11, an upper surface of the wound auxiliary positioning disc 9, and a side wall of the fixing sleeve 10, and the reinforcing plate 12 is formed as a triangular structure. Through this structural design, starting the second pen-type electric push rod 32 can push the inner arm 5 to move up and down, thereby adjusting the height of the wound auxiliary positioning disc 9. A counterweight base 2 is disposed at a bottom end of the outer arm 4, universal wheels 3 are disposed at four corners of the bottom of the counterweight base 2, and the counterweight base 2 is formed as a frustoconical structure, which is used to stably support the vertical arm 1 and facilitate movement in conjunction with the universal wheels 3.

Further, a support rail 7 is disposed on the vertical arm 1. A robotic manipulator 8 is mounted on the support rail 7, and a clamping assembly 20 capable of performing a clamping action is disposed at an end of the robotic manipulator 8. By controlling the robotic manipulator 8, the clamping assembly 20 is accurately moved to a designated position and synchronized with a surgical tool to be introduced into the patient's body, thereby achieving bone plate fixation. The support rail 7 is horizontally fixed to one side of the outer arm 4. An adjustment arm 27 is movably embedded on the support rail 7 in a length direction thereof, and the adjustment arm 27 extends through an end of the support rail 7 toward the wound auxiliary positioning disc 9. The robotic manipulator 8 is mounted on the adjustment arm 27. A lead screw 28 is rotatably mounted within the support rail 7, and the adjustment arm 27 is threadedly engaged with the lead screw 28. A second manipulation motor 29 for driving the lead screw 28 to rotate is mounted at an end of the support rail 7. A guide groove 30 is formed in each of two side walls of the adjustment arm 27 in the length direction thereof, and a protrusion 31 is disposed on each of two inner side walls of the support rail 7, each protrusion 31 is engaged with a corresponding guide groove 30. By starting the second manipulation motor 29 to drive the lead screw 28 to rotate and utilizing the threaded engagement between the lead screw 28 and the adjustment arm 27, the adjustment arm 27 can be driven to move horizontally along the support rail 7, thereby adjusting the horizontal position of the robotic manipulator 8.

In a further embodiment, the robotic manipulator 8 comprises a fixing base 15 fixed on the adjustment arm 27, a first manipulation arm 16 horizontally and rotatably mounted on the fixing base 15, a second manipulation arm 17 longitudinally and rotatably mounted on the first manipulation arm 16, a third manipulation arm 18 longitudinally and rotatably mounted on the second manipulation arm 17, and a fourth manipulation arm 19 longitudinally and rotatably mounted on the third manipulation arm 18. A first manipulation motor 21 is disposed at a rotating shaft of each of the first manipulation arm 16, the second manipulation arm 17, the third manipulation arm 18 and the fourth manipulation arm 19. Controlling the first manipulation motors 21 can drive their corresponding first manipulation arm 16, second manipulation arm 17, third manipulation arm 18 and fourth manipulation arm 19 to perform horizontal rotation and longitudinal rotation respectively. The clamping assembly 20 is disposed at the bottom end of the fourth manipulation arm 19. The clamping assembly 20 comprises a connection head 22 rotatably mounted at the bottom end of the fourth manipulation arm 19, and a pair of slide rails 23 horizontally and slidably embedded at the bottom of the connection head 22. The pair of slide rails 23 are arranged side by side. A clamping head 24 is disposed at the bottom of each slide rail 23. The pair of clamping heads 24 are symmetrically disposed. A mounting hole 26 is formed in each clamping head 24. A first pen-type electric push rod 25 for driving the slide rails 23 to move horizontally is mounted on the connection head 22. With this structural arrangement, the mounting holes 26 on the pair of clamping heads 24 can be used to mount a surgical tool to be introduced into the patient's body. The first pen-type electric push rod 25 drives the slide rails 23 to move horizontally, which can drive the pair of clamping heads 24 to bring the surgical tool closer together or apart, thereby achieving a bone plate clamping and fixation action, ensuring that the plate fits closely to the bone and provides a stable fixation effect. Such close fitting and stable fixation help promote fracture healing and reduce the occurrence of complications. By controlling the first manipulation motors 21, the corresponding first, second, third, and fourth manipulation arms 16, 17, 18, 19 can be driven to perform horizontal rotation and longitudinal rotation movements respectively, coupled with the horizontal movement adjustment of the adjustment arm 27. The high degree of automation also helps improve surgical efficiency, shorten surgical time, simplify the surgical operation process, and reduce the doctor's workload.

In a further embodiment, a control box 6 is disposed on a side wall of the outer arm 4. A circuit board and a chip are arranged inside the control box 6. An intelligent control system is arranged based on the circuit board and the chip. The intelligent control system comprises:
an image acquisition module is configured to capture, via the camera 13, image information of a patient's wound, including details such as shape, size, and depth of the wound, thereby providing
an accurate data foundation for preoperative debridement assessment and localization of the patient, the image acquisition module supporting a multi-angle shooting function to obtain complete wound information from different perspectives, allowing comprehensive understanding of wound conditions and providing support for formulating a more reasonable debridement plan;
a wireless data transmission control module is configured to employ wireless transmission technology, such as GPRS, to transmit image data captured by the camera 13 in real time to an information management center server, the information management center server has at least one IP address and stores an electronic human diagram, and upon receiving the image data, the server performs data processing and storage using TCP/IP protocol, thereby providing support for subsequent evaluation and analysis;
an artificial intelligence assessment module is configured to, based on AI technology, analyze the captured wound images and automatically assess the extent of the wound, including a wound area assessment system and a wound color assessment system, through AI recognition, patient wound conditions are recorded and archived in a timely manner; based on the extent and characteristics of the wound, the system uses an artificial intelligence algorithm to determine key debridement areas, the system simulates the debridement process, reconstructs the wound and surrounding tissues using three-dimensional (3D) modeling technology, and performs marked display based on the electronic human diagram, allowing a surgeon to perform debridement operations in a virtual environment, evaluate debridement effects in advance, and optimize a surgical plan;
and a robotic arm assistance module is configured to employ the robotic manipulator 8 as an auxiliary device, in orthopedic surgery, a surgeon uses fluoroscopy or scanning to input a patient's 3D body information into a computer, after the system automatically completes matching, the surgeon can select an appropriate surgical path, and once the surgical path is confirmed, the robotic manipulator 8 accurately moves to a specified position with sub-millimeter accuracy through camera navigation and multi-degree-of-freedom motion control technology, synchronizes with a surgical tool to be introduced into the patient's body, and achieves plate fixation of bone.

The present application, through the provision of the camera 13 which is a high-resolution camera, can clearly capture image information of a patient's wound, including details such as the shape, size, and depth of the wound, thereby providing an accurate data foundation for subsequent evaluation and localization. The multi-angle shooting function of the camera 13 can obtain complete wound information from different perspectives, allowing a more comprehensive understanding of the wound condition and providing support for formulating a more reasonable debridement plan. After acquiring the wound image information via the camera 13, a doctor can comprehensively examine the patient's wound before surgery, determine the specific location of the wound, assess the extent of the wound, and identify the key areas for debridement. After determining the wound extent and identifying the key debridement areas, the multiple groups of wound auxiliary positioning lamps 14 can be controlled to emit light beams of different colors onto the patient's wound, thereby distinguishing and marking the wound extent and the key debridement areas, which allows the doctor to perform surgical operations more precisely, reducing surgical time and trauma. Through the provision of the robotic manipulator 8 and the clamping assembly 20, the present application controls the robotic manipulator 8 to move the clamping assembly 20 accurately to a designated position and synchronize it with the surgical tool to be introduced into the patient's body, thereby achieving a bone plate clamping and fixation action, which ensures that the plate fits closely to the bone and provides a stable fixation effect. Such close fitting and stable fixation help promote fracture healing and reduce the occurrence of complications. By controlling the first manipulation motors 21, the corresponding first, second, third, and fourth manipulation arms 16, 17, 18, 19 can be driven to perform horizontal rotation and longitudinal rotation movements respectively, coupled with the horizontal movement adjustment of the adjustment arm 27. The multi-degree-of-freedom adjustment control helps improve surgical efficiency, shorten surgical time, simplify the surgical operation process, and reduce the doctor's workload. Through the provision of the control box 6, the present application utilizes artificial intelligence algorithms to simulate the debridement process and formulate surgical plans, allowing the doctor to evaluate debridement effects in advance, optimize the surgical plan, reduce surgical risks, and improve surgical precision and outcomes. Furthermore, intelligent control enables telemedicine, and the wireless data transmission control function allows doctors to evaluate the patient's wound and guide surgery remotely, providing technical support for telemedicine.

Although the embodiments of the present application have been shown and described, the specific embodiments are merely illustrative of the present application and are not intended to limit the application. The specific features, structures, materials, or characteristics described may be combined in any suitable manner in any one or more embodiments or examples. After reading this description, those skilled in the art may make modifications, substitutions, and variations to the embodiments without inventive effort as needed, but such modifications, substitutions, and variations shall be protected by patent law as long as they fall within the scope of the claims of the present application.

What is claimed is:

1. A navigation system for preoperative planning and intraoperative monitoring in a treatment of chronic osteomyelitis, comprising:
a vertical arm;
a wound auxiliary positioning disc being disposed on one side of the top end of the vertical arm;
a camera being disposed at a central position of the wound auxiliary positioning disc;
a plurality of wound auxiliary positioning lamps are disposed on a lower surface of the wound auxiliary positioning disc; wherein:
each of the wound auxiliary positioning lamps emits a monochromatic light beam;
the wound auxiliary positioning lamps are evenly arranged around the camera, a support rail is disposed on the vertical arm, a robotic manipulator is mounted on the support rail, and
a clamping assembly capable of performing a clamping action is disposed at an end of the robotic manipulator.

2. The navigation system for preoperative planning and intraoperative monitoring in the treatment of chronic osteomyelitis according to claim 1, wherein the plurality of the wound auxiliary positioning lamps are divided into at least two groups, the wound auxiliary positioning lamps of different groups emit light of different colors, and the groups of wound auxiliary positioning lamps are distributed at intervals.

3. The navigation system for preoperative planning and intraoperative monitoring in the treatment of chronic osteomyelitis according to claim 1, wherein the vertical arm comprises a vertically disposed outer arm and an inner arm movably extending through the top end of the outer arm, a second pen-type electric push rod is vertically fixed inside the outer arm, a receiving cavity for receiving the second pen-type electric push rod is formed at the bottom end inside the inner arm, and the telescopic end of the second pen-type electric push rod is connected to the inner top wall of the receiving cavity.

4. The navigation system for preoperative planning and intraoperative monitoring in the treatment of chronic osteomyelitis according to claim 3, wherein a counterweight base is disposed at a bottom end of the outer arm, universal wheels are disposed at four corners of the bottom of the counterweight base, and the counterweight base is formed as a frustoconical structure.

5. The navigation system for preoperative planning and intraoperative monitoring in the treatment of chronic osteomyelitis according to claim 3, wherein a connecting plate is horizontally fixed to one side of the wound auxiliary positioning disc, a fixing sleeve is fixed to an end of the connecting plate, the fixing sleeve is fitted over the top end of the inner arm and fixed to the inner arm by bolts, a reinforcing plate is fixed between an upper surface of the connecting plate, an upper surface of the wound auxiliary positioning disc, and a side wall of the fixing sleeve and the reinforcing plate is formed as a triangular structure.

6. The navigation system for preoperative planning and intraoperative monitoring in the treatment of chronic osteomyelitis according to claim 3, wherein the support rail is horizontally fixed to one side of the outer arm, an adjustment arm is movably embedded on the support rail in a length direction thereof, the adjustment arm extends through an end of the support rail toward the wound auxiliary positioning disc, the robotic manipulator is mounted on the adjustment arm, a lead screw is rotatably mounted within the support rail, the adjustment arm is threadedly engaged with the lead screw, a second manipulation motor for driving the lead screw to rotate is mounted at an end of the support rail, a guide groove is formed in each of two side walls of the adjustment arm in the length direction thereof, and a protrusion is disposed on each of two inner side walls of the support rail, each protrusion is engaged with a corresponding guide groove.

7. The navigation system for preoperative planning and intraoperative monitoring in the treatment of chronic osteomyelitis according to claim 6, wherein the robotic manipulator comprises a fixing base fixed on the adjustment arm, a first manipulation arm horizontally and rotatably mounted on the fixing base, a second manipulation arm longitudinally and rotatably mounted on the first manipulation arm, a third manipulation arm longitudinally and rotatably mounted on the second manipulation arm, and a fourth manipulation arm longitudinally and rotatably mounted on the third manipulation arm, a first manipulation motor is disposed at a rotating shaft of each of the first manipulation arm, the second manipulation arm, the third manipulation arm and the fourth manipulation arm, each first manipulation motor is capable of driving its corresponding manipulation arm to perform horizontal rotation and longitudinal rotation., and the clamping assembly is disposed at the bottom end of the fourth manipulation arm.

8. The navigation system for preoperative planning and intraoperative monitoring in the treatment of chronic osteomyelitis according to claim 7, wherein the clamping assembly comprises a connection head rotatably mounted at the bottom end of the fourth manipulation arm, and a pair of slide rails horizontally and slidably embedded at the bottom of the connection head, the pair of slide rails are arranged side by side, a clamping head is disposed at the bottom of each slide rail, the pair of clamping heads are symmetrically disposed, a mounting hole is formed in each clamping heads, and a first pen-type electric push rod for driving the slide rails to move horizontally is mounted on the connection head.

9. The navigation system for preoperative planning and intraoperative monitoring in the treatment of chronic osteomyelitis according to claim 3, wherein a circuit board and a chip are arranged inside the control box, an intelligent control system is arranged based on the circuit board and the chip, the intelligent control system comprises:
an image acquisition module is configured to capture, via the camera, image information of a patient's wound, comprising details such as shape, size, and depth of the wound, thereby providing an accurate data foundation for preoperative debridement assessment and localization of the patient, the image acquisition module supporting a multi-angle shooting function to obtain complete wound information from different perspectives, allowing comprehensive understanding of wound conditions and providing support for formulating a more reasonable debridement plan;
a wireless data transmission control module is configured to employ wireless transmission technology, such as GPRS, to transmit image data captured by the camera in real time to an information management center server, the information management center server has at least one IP address and stores an electronic human diagram, and upon receiving the image data, the server performs data processing and storage using TCP/IP protocol, thereby providing support for subsequent evaluation and analysis;
an artificial intelligence assessment module is configured to, based on AI technology, analyze the captured wound images and automatically assess the extent of the wound, comprising a wound area assessment system and a wound color assessment system, through AI recognition, patient wound conditions are recorded and archived in a timely manner, based on the extent and characteristics of the wound, the system uses an artificial intelligence algorithm to determine key debridement areas, the system simulates the debridement process, reconstructs the wound and surrounding tissues using three-dimensional (3D) modeling technology, and performs marked display based on the electronic human diagram, allowing a surgeon to perform debridement operations in a virtual environment, evaluate debridement effects in advance, and optimize a surgical plan;
and a robotic arm assistance module is configured to employ a robotic manipulator as an auxiliary device, in orthopedic surgery, a surgeon uses fluoroscopy or scanning to input a patient's 3D body information into a computer, after the system automatically completes matching, the surgeon select an appropriate surgical path, and once the surgical path is confirmed, the robotic manipulator accurately moves to a specified position with sub-millimeter accuracy through camera navigation and multi-degree-of-freedom motion control technology, synchronizes with a surgical tool to be introduced into the patient's body, and achieves plate fixation of bone.
